(19) United States
(12) Patent Application Publication
(10) Pub. No.: US 2016/0184000 A1
(43) Pub. Date: Jun. 30, 2016

(54) THERMAL ABLATION NEEDLE ASSEMBLY

(71) Applicant: Metal Industries Research and Development Centre, Kaohsiung (TW)

(72) Inventors: Yu-Fen Kuo, Kaohsiung (TW); Tung-Chieh Yang, Kaohsiung (TW); Tsung-Chih Yu, Kaohsiung (TW); Ho-Chung Fu, Kaohsiung (TW)

(73) Assignee: Metal Industries Research and Development Centre, Kaohsiung (TW)

(21) Appl. No.: 14/584,443

(22) Filed: Dec. 29, 2014

(51) Int. Cl. A61B 18/04

(52) U.S. Cl. CPC A61B 18/04; A61B 2018/00577

(57) ABSTRACT

A thermal ablation needle assembly includes a hollow needle body and a thermal-detecting unit. The hollow needle body has a needle tip, a receiving space formed therein, and an extension opening opposite to the needle tip and communicating the receiving space with the external environment. The thermal-detecting unit includes two wires extending into the receiving space through the extension opening. Each wire has a first end portion adjacent to the needle tip and a second end portion opposite to the first end portion. The first end portions of the wires are connected to each other and establish a short circuit.

### FIELD OF THE INVENTION

The invention relates to a thermal ablation needle assembly, more particularly to a thermal ablation needle assembly that a temperature thereof during operation can be measured.

### BACKGROUND OF THE INVENTION

As shown in FIGS. 1 and 2, a conventional thermal ablation needle assembly includes a needle 11 and a thermal-detecting unit 12 sleeved on a rear portion of the needle 11. The thermal-detecting unit 12 includes a sleeve tube 124 that, is sleeved on the rear portion of the needle 11, a connecting member 121, two wires 122 that are connected electrically to the connecting member 121, and a detecting rod 123 that extends into the sleeve tube 124, that is sleeved on the dissimilar wires 122, and that contacts a rear end of the needle 11. Each of the wires 122 has an end in proximity of the needle 11. The ends of the wires 122 are connected to each other to establish a short circuit. Thermal energy of the needle 11 is conveyed by the detecting rod 123 to the wires 122. The connecting member 121 connects electrically the wires 122 to an external electronic device for determining the temperature of the needle 11 based on signals read off the wires 122 that are associated with the thermal energy conveyed thereto. As such, a user can effectively know the temperature of the needle 11 during operation of the conventional thermal ablation needle assembly instead of relying only on the experience of the user in determination of the temperature of the needle 11.

However, the structure of the conventional thermal ablation needle assembly is relatively complicated, which results in a relatively high manufacturing cost. Moreover, since the detecting rod 123 contacts the rear end of the needle 11, the temperature of the tip of the needle 11 cannot be accurately determined.

### SUMMARY OF THE INVENTION

Therefore, the object of the present invention is to provide a thermal ablation needle assembly that can alleviate the drawbacks associated with the abovementioned prior art.

Accordingly, a thermal ablation needle assembly of the present invention includes a hollow needle body and a thermal-detecting unit. The hollow needle body has a needle tip, a receiving space that is formed therein, and an extension opening that is opposite to the needle tip and that communicates the receiving space with the external environment. The thermal-detecting unit includes two wires that extend into the receiving space of the hollow needle body through the extension opening. Each of the wires has a first end portion that is adjacent to the needle tip. The first end portions of the wires are connected to each other and establish a short circuit for detecting thermal energy from the needle tip. Each of the wires further has a second end portion that is opposite to the first end portion. The second end portions of the wires are for transmitting signals associated with the detected thermal energy.

### BRIEF DESCRIPTION OF THE DRAWINGS

Other features and advantages of the present invent ion will become apparent in the foil owing detailed description of the embodiments with reference to the accompanying drawings, of which:

FIG. 1 is a perspective view of a conventional thermal ablation needle assembly;

FIG. 2 is a side view of the conventional thermal ablation needle assembly;

FIG. 3 is a fragmentary sectional view of a first embodiment of a thermal ablation needle assembly according to the invention;

FIG. 4 is a fragmentary sectional view of a second embodiment of the thermal ablation needle assembly according to the invention;

FIG. 5 is a fragmentary sectional view of a third embodiment of the thermal ablation needle assembly according to the invention;

FIG. 6 is a fragmentary sectional view of a fourth embodiment of the thermal ablation needle assembly according to the invention;

FIG. 7 is a sectional view of a hollow needle body and an insertion unit of the fourth embodiment;

FIG. 8 is a fragmentary sectional view of a fifth embodiment of the thermal ablation needle assembly according to the invention;

FIG. 9 is a sectional view of the hollow needle body and the insertion unit of the fifth embodiment;

FIG. 10 is a fragmentary sectional view of a modification of the third embodiment;

FIG. 11 is a fragmentary sectional view of a modification of the fourth embodiment;

FIG. 12 is a fragmentary sectional view of a sixth embodiment of the thermal ablation needle assembly according to the invention; and

FIG. 13 is a sectional view of the hollow needle body and the insertion unit of the fourth embodiment.

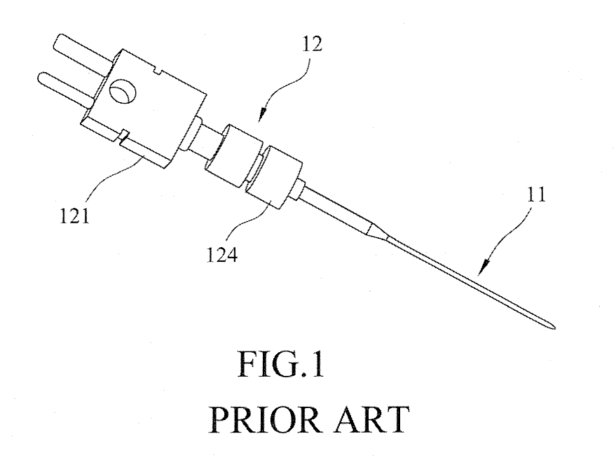

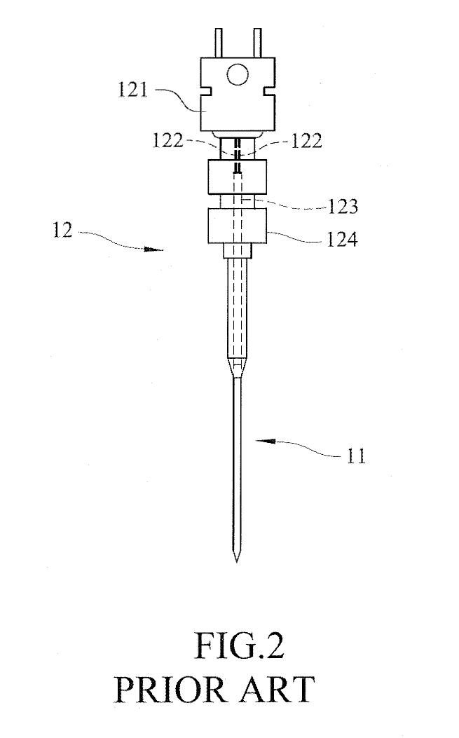

### DETAILED DESCRIPTION OF THE EMBODIMENTS

Before the present invention is described in greater detail, it should be noted that like elements are denoted by the same reference numerals throughout the disclosure.

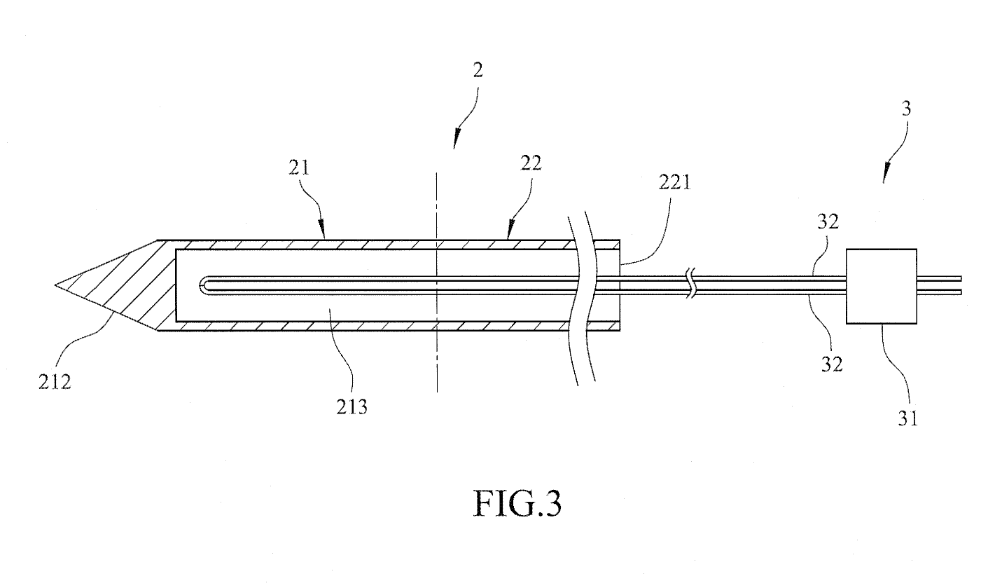

As shown in FIG. 3, a first embodiment of a thermal ablation needle assembly according to the present invention includes a hollow needle body 2 and a first thermal-detecting unit 3.

The hollow needle body 2 defines a receiving space 213 and includes a magnetic induction segment 21 formed with a needle tip 212, and a nonmagnetic induction segment 22 extending rearwardly from a rear end of the magnetic induction segment 21 and formed with an extension opening 221 that is opposite to the needle tip 212 and that communicates the receiving space 213 with the external environment. The receiving space 213 extends through the nonmagnetic induction segment 22 into the magnetic induction segment 21. The first thermal-detecting unit 3 includes two first wires 32 that are dissimilar to each other and that extend into the receiving space 213 of the hollow needle body 2 through the extension opening 221, and a first connecting member 31 adapted to connect electrically the first wires 32 to an external temperature-determining device (not shown). Each of the first wires 32 has a first end portion that is adjacent to the needle tip 212 and that are adapted for detecting thermal energy from the needle tip 212. The first end portions of the first wires 32 are connected to each other and establish a short circuit. Each of the first wires 32 further has a second end portion that is opposite to the first end portion for transmitting a signal associated with the thermal energy detected by the first end portion to the external temperature-determining device. In this embodiment, the first wires 32 are provided with an electrically-insulating coating (not shown) that prevents short circuiting between parts of the first wires 32 other than the first end portions. It should be noted that the first connecting member 31 is not an essential component of this invention when the first wires 32 are able to establish electrical connection with the external temperature-determining device directly.

The first thermal-detecting unit 3 and the temperature-determining device utilize the thermoelectric effect to determine the temperature of the thermal ablation needle assembly, where the signals at the second end portions of the first, wires 32 represent a potential difference resulting from a temperature differential between the first end portions of the first wires 32 as the thermal energy at the needle tip 212 is conducted differently by the two dissimilar first wires 32, and the temperature-determining device determines the temperature of the thermal ablation needle assembly based on the potential difference. In use, since the short circuit occurs at a position near the needle tip 212 of the magnetic induction segment 21, the temperature determined closely represents that of the needle tip 212. This enables a user to keep abreast of the temperature of the magnetic induction segment 21 inserted in a target area, making it easy for the user to avoid unnecessary damage to healthy tissues around the target area by maintaining the heat at an optimal temperature.

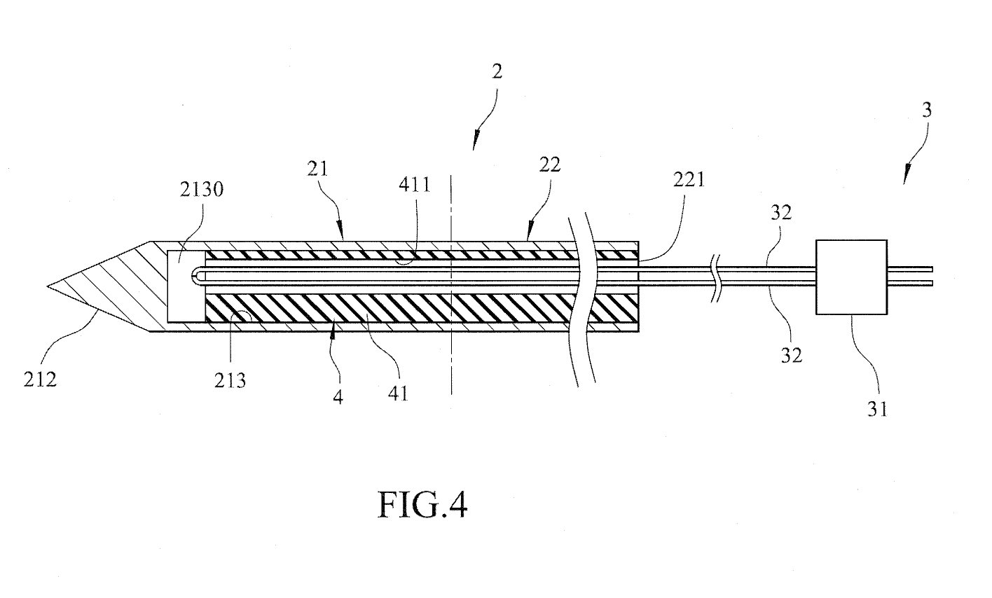

Referring to FIG. 4, a second embodiment of the thermal ablation needle assembly according to the present invention is similar to the first embodiment, except that, the thermal ablation needle assembly of the second embodiment further includes an insertion unit 4 inserted into the receiving space 213. The insertion unit 4 includes an insertion member 41 that is formed with a first through hole 411. The two first wires 32 extend through the first through hole 411. During assembly, the first wires 32 are first extended through the first through hole 411 of the insertion member 41, then inserted into the receiving space 213 along with the insertion member 41, thereby improving the convenience of the assembly process. It should be noted that, in use, the insertion member 41 may either completely fill the receiving space 213 or leave a space 2130 proximate to the needle tip 212 so that a white spot may be presented on a sonogram (not shown) generated by an ultrasound scanner to track movement of the hollow needle body 2. Therefore, the second embodiment not only has the advantages of the first embodiment, but grants convenience and accuracy to the assembly and operation processes of the present invention.

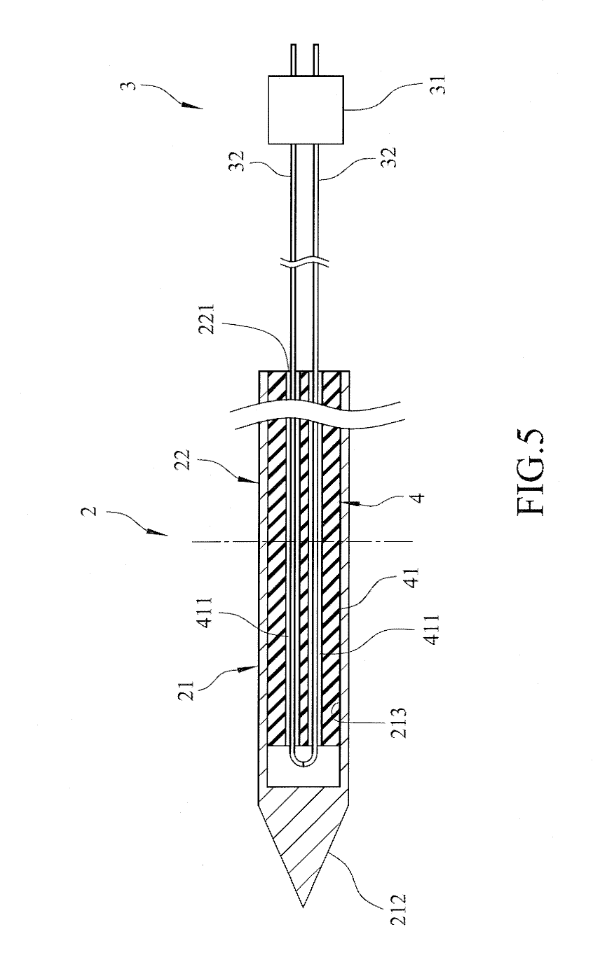

Referring to FIG. 5, a third embodiment of the thermal ablation needle assembly according to the present invention is similar to the second embodiment, except that the insertion member 41 of this embodiment is made of an electrically-insulating material and the insertion member 41 is formed with two first through holes 411. Daring assembly, the two first wires 32 are first extended respectively through the two first through holes 411 before forming the short circuit, then inserted into the receiving space 213 along with the insertion member 41. Therefore, the third embodiment not only has the advantages of the second embodiment, but prevents the short circuit from occurring at undesired positions of the first wires 32 by virtue of the electrically-insulating material should the first wires 32 be bare.

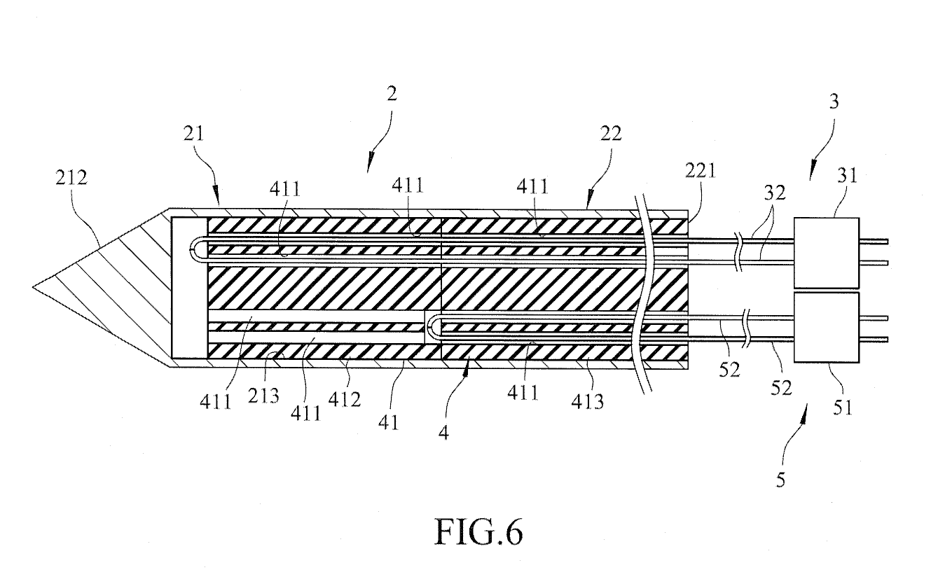

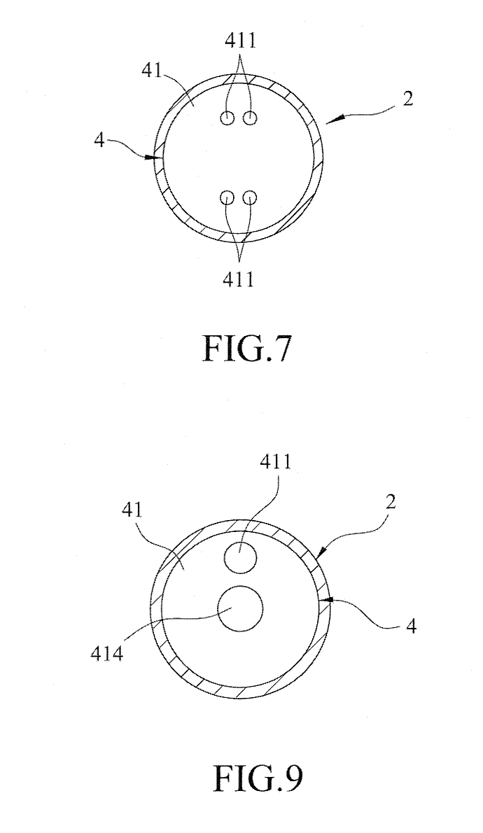

Referring to FIGS. 6 and 7, a fourth embodiment of the thermal ablation needle assembly according to the present invention is similar to the third embodiment, except that the thermal ablation needle assembly of this embodiment further includes a second thermal-detecting unit 5, and that the insertion member 41 is of a two-piece design having first and second insertion portions 412, 413 that are connected to each other and that are disposed respectively proximate to and distal from the needle tip 212. In this embodiment, each of the first and second insertion portions 412, 413 is formed with four first through holes 411 disposed in a manner as illustrated by FIG. 7. For the sake of clarity, the four first through holes 411 of each of the first and second insertion portions 412, 413 are illustrated to be spaced apart along a straight line in FIG. 6. The second thermal-detecting unit includes two second wires 52 that are dissimilar to each other and that extend into the receiving space 213 of the hollow needle body 2 through the extension opening 221, and a second connecting member 51 that is adapted to connect electrically the second wires 52 to an external temperature-determining device (not shown). Each of the second wires 52 has a first end portion that is distal from the needle tip 212. The first end portions of the second wires 52 are connected to each other and establish a short circuit. Each of the second wires 52 further has a second end portion that is opposite to the first end portion thereof for transmitting a signal to the external temperature-determining device. During assembly, the two second wires 52 are extended respectively through two of the first through holes 411 of the second insertion portion 413 before forming the short circuit, and the two first wires 32 are first extended respectively through the other two of the first through holes 411 of the second insertion portion 413, then extended respectively through two of the first through holes 411 of the first insertion portion 412 before forming the short circuit. The insertion member 41 is then inserted into the receiving space 213 along with the first and second wires 32, 52. In this way, the short circuit of the first wires 32 occurs at a posit ion proximate to the needle tip 212, while the short circuit of the second wires 52 occurs at a juncture of the first and second insert ion port ions 412, 413, thereby allowing the temperature of different sections of the thermal ablation needle assembly to be measured so that the user may, during operation, accordingly make any adjustments necessary. It should be noted that, while the division of the magnetic induction segment 21 and the nonmagnetic induction segment 22 of the first embodiment is formed by a difference in the compositional properties of the hollow needle body 2 (see FIG. 3), the division in this embodiment is formed by the difference in compositional properties of the first and second insertion portions 412, 413, e.g., the first insertion portion 412 may be made of a high magnetic permeability material, such as ferrite, and the second insertion portion 413 may be made of a magnetically impermeable material, such as magnesium oxide or zirconium oxide. Therefore, this embodiment provides a different configuration for forming the magnetic induction segment 21 and the nonmagnetic induction segment 22.

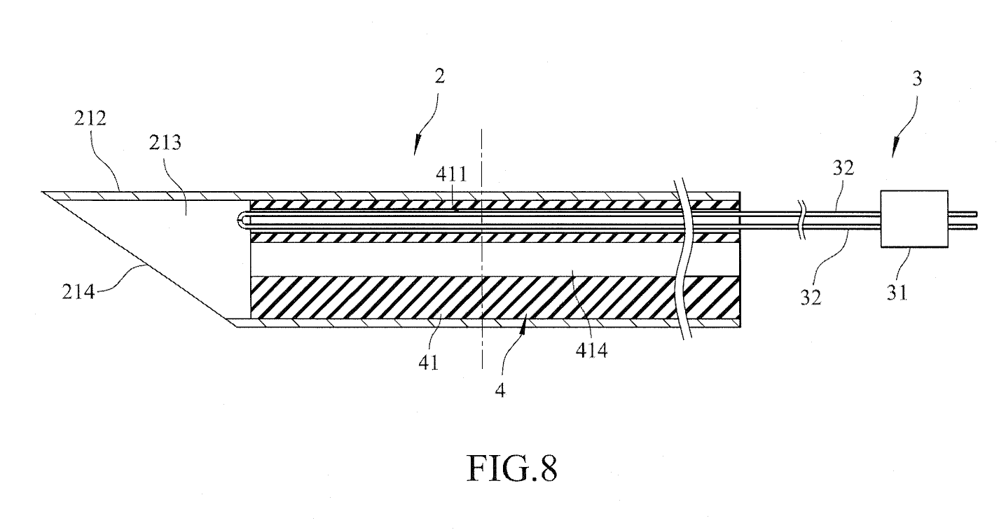

Referring to FIGS. 8 and 9, a fifth embodiment of the thermal ablation needle assembly according to the present invention is similar to the second embodiment, except that the insertion member 41 further has a second through hole 414 spaced apart from the first through hole 411 and being in spatial communication with the receiving space 213, and that the needle tip 212 is formed with an injection opening 214 being in spatial communication with the receiving space 213. The receiving space 213 of this embodiment extends all the way to the needle tip 212. In this way, the user is not only able to conduct ablation of the target area with the thermal ablation needle assembly, but also inject drugs and extract tissue therewith by virtue of the second through hole 414. It should be noted that the structure of the thermal ablation needle assembly disclosed in FIGS. and 6 may also be configured to have the second through hole 414 and the injection opening 214, as illustrated in FIGS. and 11, to confer the foregoing advantages.

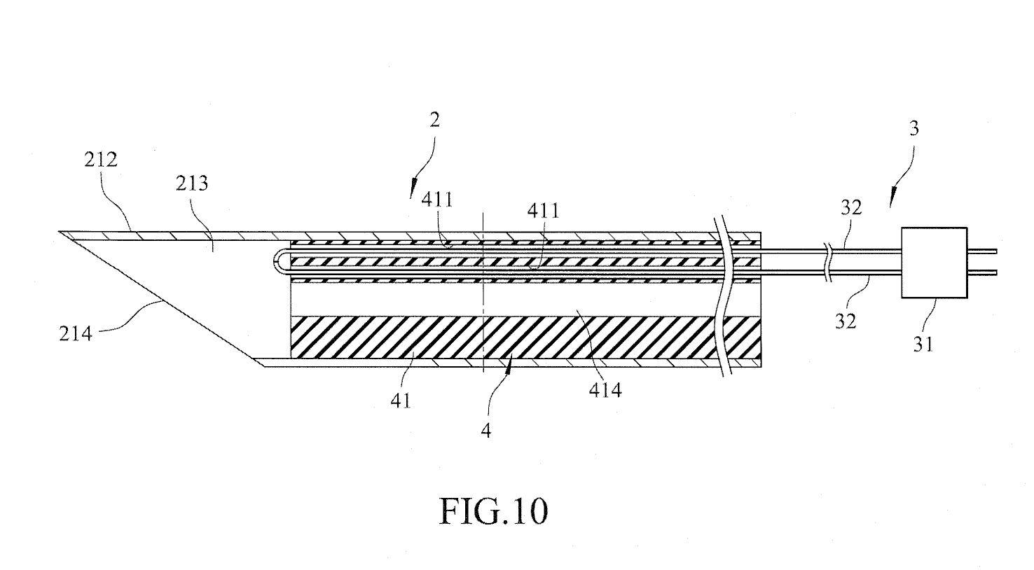

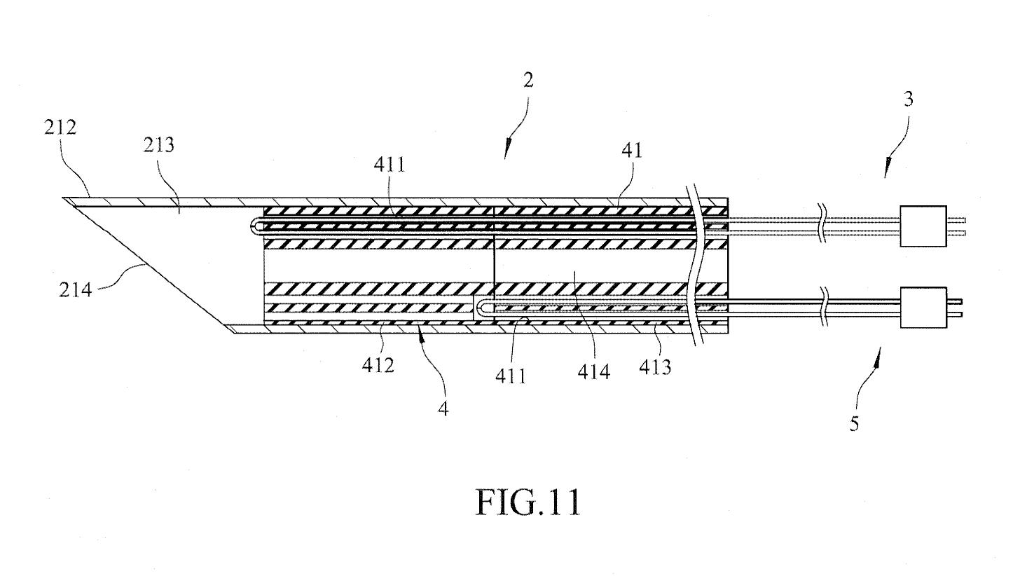

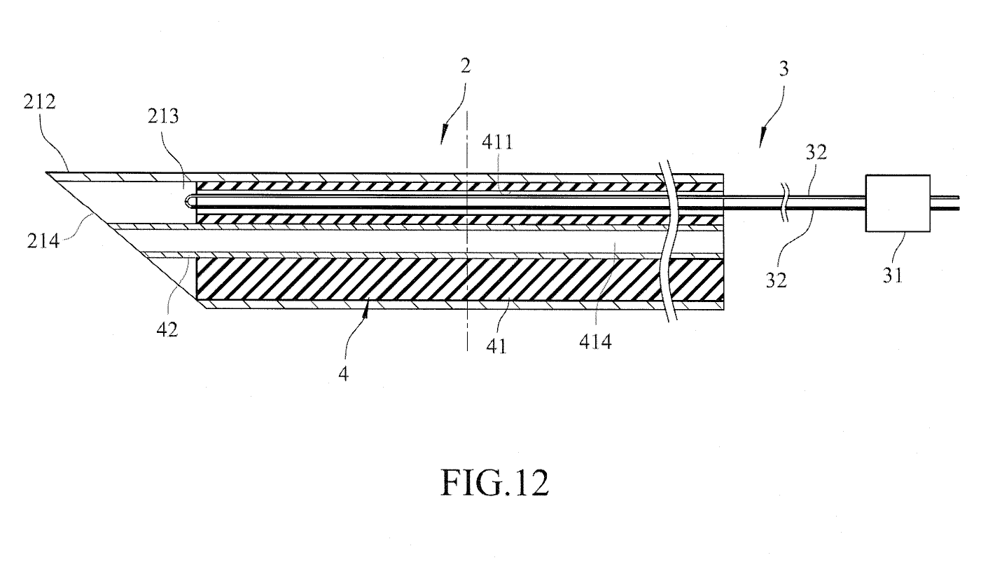

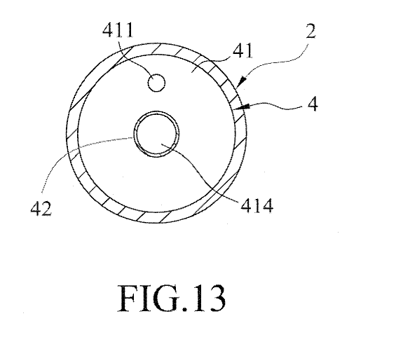

Referring to FIGS. 12 and 13, a sixth embodiment of the thermal ablation needle assembly according to the present invention is similar to the fifth embodiment, except that the insertion unit 4 further includes a tubular protection member 42 that is fixed to an inner surface of the insertion member 41 defining the second through hole 414 and that extends along the second through hole 414. Therefore, the thermal ablation needle assembly of this embodiment is formed with an inner space and an outer space, with the first wires 32 disposed in the outer space between the hollow needle body 2 and the tubular protection member 42. Thus, when a drug is injected through the second through hole 414, the tubular protection member 42 prevents the drug from spreading or seeping into the insertion member 41 and compromising the electrical-insulating capacity thereof. The tubular protection member 42 of this embodiment is made of metal or ceramic material, or any other material that can prevent drugs from seeping. It should be noted that the structure of the thermal ablation needle assembly disclosed in FIGS. 8, 10 and 11 may also be configured to have the tubular protection member 42 to confer the foregoing advantages.

In sum, a thermal ablation needle assembly that a temperature thereof during operation can be measured is not only configured to have a more streamlined structure, but also allows a user to keep track of a current temperature during operation by having the first wires 32 of the first thermal-detecting unit 3 establish a short circuit at a position near the needle tip 212, enabling the user to adjust the heat to an optimal temperature for ablation so that any unnecessary damage to tissue may be avoided.

While the present invention has been described in connection with what are considered the most practical embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A thermal ablation needle assembly comprising:
a hollow needle body having a needle tip, a receiving space that is formed therein, and an extension opening that is opposite to said needle tip and that communicates said receiving space with the external environment;
a first thermal-detecting unit including two first wires that extend into said receiving space of said hollow needle body through said extension opening, each of said first wires having a first end portion that is adjacent to said needle tip, said first end portions of said first wires being connected to each other and establishing a short circuit for detecting thermal energy from said needle tip, each of said first wires further having a second end portion that is opposite to said first end portion, said second end portions of said first wires being for transmitting signals associated with the detected thermal energy.

2. The thermal ablation needle assembly as claimed in claim 1, wherein said hollow needle body includes a magnetic induction segment formed with said needle tip, and a nonmagnetic induction segment connected to said magnetic induction segment and formed with said extension opening.

3. The thermal ablation needle assembly as claimed in claim 1, further comprising a second thermal-detecting unit that includes two second wires extending into said receiving space of said hollow needle body through said extension opening, each of said second wires having a first end portion that is distal from said needle tip, said first end portions of said second wires being connected to each other and establishing a short circuit.

4. The thermal ablation needle assembly as claimed in claim 3, further comprising an insertion unit that is inserted into said receiving space and that includes an insertion member, said insertion member having first and second insertion portions that are connected to each other and that are disposed respectively proximate to and distal from said needle tip, each of said first and second insertion portions being formed with a plurality of first through holes, said first wires extending respectively through two of said first through holes of said second insertion portion and extending respectively through two of said first through holes of said first insertion portion, said second wires extending respectively through the other two of said first through holes of said second insertion portion.

5. The thermal ablation needle assembly as claimed in claim 4, wherein said insertion member further has a second through hole extending through said first and second insertion portions, spaced apart from said first through holes, and being in spatial communication with said receiving space, said needle tip of said hollow needle body being formed with an injection opening that is in spatial communication with said receiving space.

6. The thermal ablation needle assembly as claimed in claim 5, wherein said insertion member further having an inner surface that defines said second through hole, said insertion unit further including a tubular protection member that is fined to said inner surface and that extends along said second through hole.

7. The thermal ablation needle assembly as claimed in claim 6, wherein said protection member is made of metal or a ceramic material.

8. The thermal ablation needle assembly as claimed in claim 4, wherein said insertion member is made of an electrically-insulating material.

9. The thermal ablation needle assembly as claimed in claim 4, wherein;
said first thermal-detecting unit further includes a first connecting member adapted to connect electrically said first wires to an external temperature-determining device; and
said second thermal-detecting unit further includes a connecting member adapted to connect electrically said second wires to an external temperature-determining device.

10. The thermal ablation needle assembly as claimed in claim 4, wherein said first insertion portion of said insertion member is made of a high magnetic permeability material, and said second insertion portion of said insertion member is made of a magnetically impermeable material.

11. The thermal ablation needle assembly as claimed in claim 3, wherein each of said first and second wires is provided with an electrically-insulating coating.

12. The thermal ablation needle assembly as claimed in claim 1, further comprising an insertion unit inserted into said receiving space and including an insertion member that is formed with a first through hole, said first wires extending through said first through hole.

13. The thermal ablation needle assembly as claimed in claim 12, wherein said insertion member further has a second through hole that is spaced apart from said first through hole and that is in spatial communication with said receiving space, said needle tip of said hollow needle body being formed with an injection opening that is in spatial communication with said receiving space.

14. The thermal ablation needle assembly as claimed in claim 13, wherein said insertion member further having an inner surface that defines said second through hole, said insertion unit further including a tubular protection member that is fixed to said inner surface and that extends along said second through hole.

15. The thermal ablation needle assembly as claimed in claim 14, wherein said protection member is made of metal or a ceramic material.

16. The thermal ablation needle assembly as claimed in claim 12, wherein said insertion member is made of an electrically-insulating material.

17. The thermal ablation needle assembly as claimed in claim 1, wherein said first thermal-detecting unit further includes a first connecting member adapted to connect electrically said first wires to an external temperature-determining device.
